(19) United States
(12) Patent Application Publication
(10) Pub. No.: US 2022/0351862 A1
(43) Pub. Date: Nov. 3, 2022

(54) PREDICTION OF THE ONSET OF CRITICAL LIMB THREATENING ISCHEMIA (CLTI)

(71) Applicant: Baylor College of Medicine, Houston, TX (US)

(72) Inventors: Jayer Chung, Houston, TX (US); Joseph L. Mills, SR., Houston, TX (US)

(21) Appl. No.: 17/661,135

(22) Filed: Apr. 28, 2022

Related U.S. Application Data
(60) Provisional application No. 63/201,415, filed on Apr. 28, 2021.

(51) Int. Cl. G16H 50/30; G16H 50/70; G06N 20/00

(52) U.S. Cl. CPC G16H 50/30; G16H 50/70; G06N 20/00

(57) ABSTRACT

Prediction of a baseline risk of major amputation and wound healing and other healthcare outcomes associated with chronic limb threatening ischemia (CLTI) may be determined using a combination of two-dimensional (2-D) perfusion angiography results from before and/or after percutaneous intervention with a Wound Ischemia foot Infection (WIfI) Score, such as using a machine learning algorithm. The combination of 2-D perfusion angiography and WIfI score enables precise prediction of the baseline risk of major amputation and wound healing associated with chronic limb threatening ischemia (CLTI). This score may be used to stratify limbs by their baseline risk of major amputation with and without therapy.

### CROSS-REFERENCE TO RELATED PATENT APPLICATIONS

This application claims the benefit of priority of U.S. Provisional Patent Application No. 63/201,415 filed on Apr. 28, 2021 entitled “PREDICTION OF THE ONSET OF CRITICAL LIMB THREATENING ISCHEMIA (CLTI),” the disclosure of which is incorporated by reference in its entirety.

### FIELD OF THE DISCLOSURE

The instant disclosure relates to methods and information handling systems for healthcare intelligence and analytics for augmented decision making. More specifically, portions of this disclosure relate to monitoring and analyzing healthcare data to predict healthcare outcomes such as an onset of Critical Limb Threatening Ischemia (CLTI).

### BACKGROUND

There is an inability to accurately predict adequacy of blood supply in patients with Critical Limb Threatening Ischemia (CLTI). Currently, physicians aim to maximize the amount of blood flow using endovascular (balloons, atherectomy and/or stents) or open surgical (bypass, endarterectomy) techniques. Unfortunately, the outcome of any of these techniques on a patient is unpredictable and physicians often unsuccessfully try one or more of these procedures without improving the patient's outcome. Indeed, some of these techniques have a high failure rate, with major amputation and/or death occurring in as much as 20% of limbs at one year from the intervention. Repeat procedures and high hospital utilization frequently occur as patients and physicians both attempt to save the limb. This incurs significant cost, and burden, to the patient, hospital, and society as a whole.

### SUMMARY

Prediction of a baseline risk of major amputation and wound healing and other healthcare outcomes associated with chronic limb threatening ischemia (CLTI) may be determined using a combination of two-dimensional (2-D) perfusion angiography results from before and/or after percutaneous intervention with a Wound Ischemia foot Infection (WIfI) Score. The combination of 2-D perfusion angiography and WIfI score enables precise prediction of the baseline risk of major amputation and wound healing associated with chronic limb threatening ischemia (CLTI). This score may be used to stratify limbs by their baseline risk of major amputation with and without therapy. The addition of 2-D perfusion angiography will permit accurate calibration of risk-prediction algorithm. In some embodiments, the combination of 2-D perfusion angiography and WIfI score of patients with known outcomes, sometimes in combination with other healthcare records for the patient, may be used to train a machine learning algorithm. The machine learning algorithm may then be used to predict outcomes of patients by inputting a patient's WIfI score and 2-D perfusion angiography results to the machine learning algorithm and receiving a predicted outcome determined by the algorithm based on relationships between the WIfI scores and 2-D perfusion angiography results identified in the training data during the training of the algorithm. The training of the machine learning algorithm may be supplemented by other healthcare data regarding the patient, when available, and likewise used by the algorithm in predicting outcomes for patients.

The use of 2-D angiography, when appropriately stratified by WIfI, enables physicians and caregivers to ascertain when adequate perfusion has been achieved with a given revascularization. By the same token, 2-D angiography may also assist in clarifying when further attempts at revascularization are futile. This is especially critical to prevent unnecessary procedures, hospitalization, and patient suffering. Embodiments of this disclosure, identify parameters obtained via 2-D perfusion angiography that predict wound healing and limb salvage outcomes in the context of their individual WIfI presentation.

To measure, analyze, and determine the blood flow through vessels and/or to a wound, an information handling system may use an algorithm that may include programmable rule(s), such as a machine learning algorithm. The information handling system may receive healthcare data such as a Wound Ischemia foot Infection (WIfI) score, a 2-D perfusion angiography scan (or other data relating to the scan), and/or healthcare records for a patient. With the healthcare data, the information handling system may determine a risk factor for the patient with increased precision, reliability, integration, and/or numerical literacy. For example, the information handling system may use the WIfI score to calibrate the 2-D perfusion angiography. A calibrated 2-D perfusion angiography scan may provide physicians and patients with an healthcare risk factor and data. Identifying the appropriate use and procedures of the healthcare data such as the Will score, 2-D perfusion angiography scan, and patient healthcare factors may save the limbs and lives of Critical Limb Threatening Ischemia (CLTI) patients. With the healthcare data, the information handling system may determine the risk factor which may include a non-numerical or numerical value such as an onset of Critical Limb Threating Ischemia (CLTI), a baseline risk for major amputation, wound healing, and/or death. With a numerical or non-numerical value, the physician and patient may be better informed of the healthcare outcomes and the best course of action to provide augmented decision making.

Additionally, the appropriate use and procedures for the healthcare data may increase positive healthcare outcomes and utilization. For example, physicians may have to repeat procedures, which may result additional hospital therapies or treatments and inadequate utilization of hospital resources. A calibrated 2-D perfusion angiography based on the WIfI score may also assist in clarifying when further attempts at revascularization are futile and preventing unnecessary procedures, hospitalization and suffering. As the physicians and patients make decisions based on the risk factor, the financial burden to the patient, hospital, and society may be reduced.

According to one embodiment, a method may include receiving, by an information handling system, a Wound Ischemia foot Infection (WIfI) score for a patient; receiving, by an information handling system, 2-D perfusion angiography scan data for the patient, wherein the 2-D perfusion angiography scan is calibrated based on the Wound Ischemia foot Infection (WIfI) score for the patient; analyzing the Wound Ischemia foot Infection (WIfI) score for the patient and the 2-D perfusion angiography scan data for the patient; and determining a risk factor for the patient. In certain embodiments, the method may include additional steps for receiving, by an information handling system, a healthcare record for the patient; analyzing, by the information handling system, the Wound Ischemia foot Infection (WIfI) score for the patient, the 2-D perfusion angiography scan for the patient, and the healthcare record for the patient; and determining, by the information handling system, the risk factor for the patient.

According to some embodiment, the method may include analyzing, by the information handling system, the 2-D perfusion angiography scan for the patient including identifying at least one of a peak intensity to wound, a rate to measure baseline, a plateau at peak intensity, area under a curve of the scan, and a speed dissipation of a signal.

In certain embodiments, the method may further include analyzing, by the information handling system, the healthcare record for the patient including identifying clinical conditions and classifications of the patient. In another embodiment, the method may further include determining, by the information handling system, the risk factor for the patient including calculating and displaying a risk of at least one of an onset of Critical Limb Threating Ischemia (CLTI), a major amputation, a wound healing, and death.

The method may be embedded in a computer-readable medium as computer program code comprising instructions that cause a processor to perform the steps of the method. In some embodiments, the processor may be part of an information handling system.

As used herein, angiographic perfusion imaging refers to a post-processing modality for visualizing the inside, or lumen, of blood vessels, including arteries and/or veins, which may be performed without contrast or radiation, although it does not exclude imaging obtained with contrast or radiation. Angiographic perfusion may provide more information about perfusion status and microcirculation of the foot.

The foregoing has outlined rather broadly certain features and technical advantages of embodiments of the present invention in order that the detailed description that follows may be better understood. Additional features and advantages will be described hereinafter that form the subject of the claims of the invention. It should be appreciated by those having ordinary skill in the art that the conception and specific embodiment disclosed may be readily utilized as a basis for modifying or designing other structures for carrying out the same or similar purposes. It should also be realized by those having ordinary skill in the art that such equivalent constructions do not depart from the spirit and scope of the invention as set forth in the appended claims. Additional features will be better understood from the following description when considered in connection with the accompanying figures. It is to be expressly understood, however, that each of the figures is provided for the purpose of illustration and description only and is not intended to limit the present invention.

### BRIEF DESCRIPTION OF THE DRAWINGS

For a more complete understanding of the disclosed system and methods, reference is now made to the following descriptions taken in conjunction with the accompanying drawings:

FIG. 1 is an illustration of data processing in a computer network according to some embodiments of the disclosure.

FIG. 2 is a table illustrating example inputs and outputs to the information handling system according to some embodiments of the disclosure.

FIG. 3 is a flow chart illustrating a method according to some embodiments of the disclosure.

FIG. 4 is a schematic block diagram illustrating an information handling system according to some embodiments of the disclosure.

FIG. 5 is a schematic block diagram illustrating an information handling system according to some embodiments of the disclosure.

### DETAILED DESCRIPTION

An information handling system may execute an algorithm to receive healthcare data and/or to analyze healthcare data corresponding to blood flow through vessels and/or to a wound, with the algorithm providing an output, such as a recommended procedure (e.g., therapy or amputation) and/or predicted outcomes (e.g., risk of amputation or would healing) for one or more procedures. The algorithm may include programmable rule(s) that are determined based on training data and/or identifying trends in healthcare records between the procedures and patient data. Using the algorithm, the information handling system may receive healthcare data such as a Wound Ischemia foot Infection (WIfI) score, a 2-D perfusion angiography scan data, and/or a healthcare record for a patient. and may determine a risk factor for the patient with increased precision, reliability, integration, and/or numerical literacy. The risk factor may include a non-numerical or numerical value indicating an onset of Critical Limb Threating Ischemia (CLTI), a baseline risk for major amputation, wound healing, and/or death. The algorithm may analyze the healthcare data such as the WIfI score, 2-D perfusion angiography scan data, and patient healthcare records based on medical usage and procedures for Critical Limb Threatening Ischemia (CLTI) patients. With a numerical or non-numerical value for the risk factor, the physician and patient may be informed of the healthcare risks and outcomes. For example, the use of 2-D perfusion angiography scan data, when appropriately stratified by WIfI, may enable physicians and caregivers to ascertain when adequate perfusion has been achieved with a given revascularization.

The algorithm may be a non-linear regression model, linear regression model, or machine learning algorithm. Machine learning models, as described herein, may include logistic regression techniques, linear discriminant analysis, linear regression analysis, artificial neural networks, machine learning classifier algorithms, or classification/regression trees in some embodiments. In some aspects, the machine learning may include one or more artificial neural networks, which may include an interconnected group of artificial neurons (e.g., neuron models) for modeling relationships between parameters, such as 2-D perfusion angiography scan data and WIfI score. In some aspects, the machine learning may include one or more convolutional neural networks, which are a type of feed-forward artificial neural network. Convolutional neural networks may include collections of neurons that each have a receptive field and that collectively tile an input space. In some aspects, the machine learning may include one or more deep learning architectures, such as deep belief networks and deep convolutional networks, which are layered neural networks architectures in which the output of a first layer of neurons becomes an input to a second layer of neurons, the output of a second layer of neurons becomes and input to a third layer of neurons, and so on. Deep neural networks may be trained to recognize a hierarchy of features. In various aspects, machine learning systems may employ Naive Bayes predictive modeling analysis of several varieties, learning vector quantization, or implementation of boosting algorithms such as Adaboost or stochastic gradient boosting systems for iteratively updating weighting to train a machine learning classifier to determine a relationship between an influencing attribute, such as WIfI score and 2-D perfusion angiography scan data, and an outcome, such as a predicted outcome of a procedure, a risk factor, or other outputs described herein, and/or a degree to which such an influencing attribute affects the outcome of such a system.

The following example embodiments describe and illustrate various features and descriptions of how the invention is integrated into an algorithm and information handling system and how it is an improvement of methods and processes used in a healthcare setting.

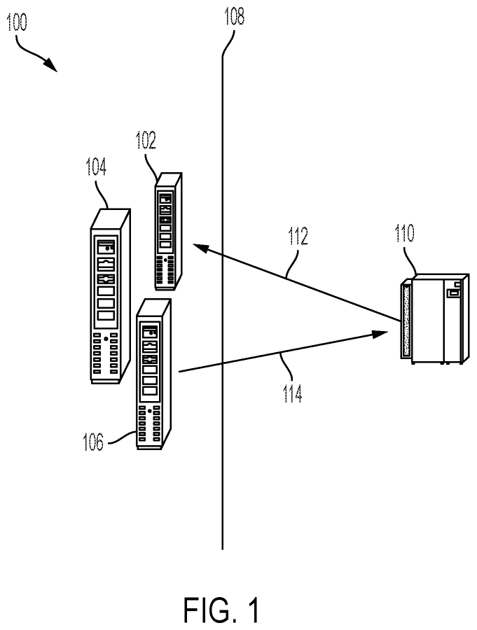

FIG. 1 is an illustration of data processing in a computer network according to some embodiments of the disclosure. Systems 102, 104, 106, and 110 may include a server, a handheld device such as a tablet or phone, or the like to process healthcare data. The systems 102, 104, 106, and 110 may provide healthcare intelligence and analytics. In some embodiments, the server 102, server 104, and server 106, may be remote from server 110 with a separation 108 that may be geographic in nature or virtual in nature (such as with a firewall or network boundary). The systems 102, 104, 106 may be configured to provide healthcare data, including records, WIfI scores, and/or 2-D perfusion angiography data through a network connection 114 and to receive a risk score through network connection 112. The network connections 112 and 114 may include hardwired connections or non-hardwired connections, including a local area network (LAN), wide area network (WAN), and/or the Internet. The healthcare inputs may include healthcare data related to the Wound Ischemia foot Infection (WIfI) score, a 2-D perfusion angiography scan, and/or a healthcare record for a patient. Details of the Wound Ischemia foot Infection (WIfI) score are described in Joseph L. Mills, “Update and validation of the Society for Vascular Surgery wound, ischemia, and foot infection threatened limb classification system,” Seminars in Vascular Surgery 27(1), pp. 16-22 (2014), and L. X. Zhan et al., “The Society for Vascular Surgery (SVS) lower extremity threatened limb classification system based on wound, ischemia, and foot infection (WIfI) correlates with risk of major amputation and time to wound healing,” J Vasc Surg 61, pp. 939-944 (2015), which are incorporated by reference herein. A WIfI score as described herein may be the original, updated, or other variation of the WIfI score described in these references. System 110 may process the collected data to predict an outcome and provide that information to one or more of the systems 102, 140, and/or 106. The healthcare outputs may include a non-numerical or numerical value such as a prediction of onset of Critical Limb Threating Ischemia (CLTI), baseline risk for major amputation, wound healing, and/or death. In some embodiment, different configurations of the systems 102, 104, and 106 may be implemented for determining patient outcome, such as when the processing is performed on a single information handling system. Although a server-client organization for information handling systems is described in FIG. 1, the operation of a machine learning algorithm based on WIfI score and angiography data to determine a patient outcome or recommendation may also be implemented on a single information handling system, such as a single computer or a single mobile device.

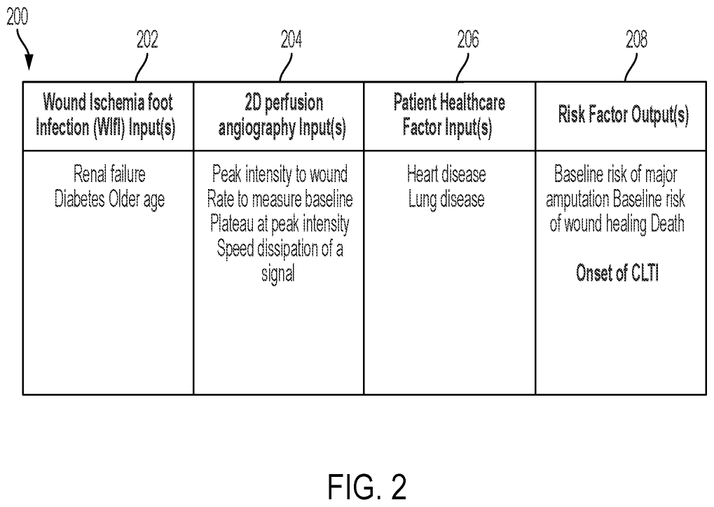

FIG. 2 illustrates a table 200 of healthcare inputs and outputs to the information handling system according to some embodiments of the disclosure. A Wound Ischemia foot Infection (WIfI) score may be input from patient healthcare records and/or determined from information in the records including records of renal failure, diabetes, and/or age. 2-D perfusion angiography input(s) 204 may include a peak intensity to wound, a rate to measure baseline, a plateau to peak intensity, and/or a speed of dissipation of a signal measured during the 2-D perfusion angiography. The inputs 204 may be from 2-D perfusion angiography performed prior to or after percutaneous coronary intervention and the data corresponding to inputs 204 marked as corresponding to the before or after situation. Patient healthcare factor input(s) 206 may include indications of heart disease and/or lung disease from healthcare records. An information handling system may receive the Wound Ischemia foot Infection (WIfI) input(s) 202, the 2-D perfusion angiography input(s) 204, and the patient healthcare factor input(s) 206 to determine risk factor output(s) 208, such as an onset of Critical Limb Threating Ischemia (CLTI), a baseline risk of major amputation, baseline risk of wound healing, and/or death.

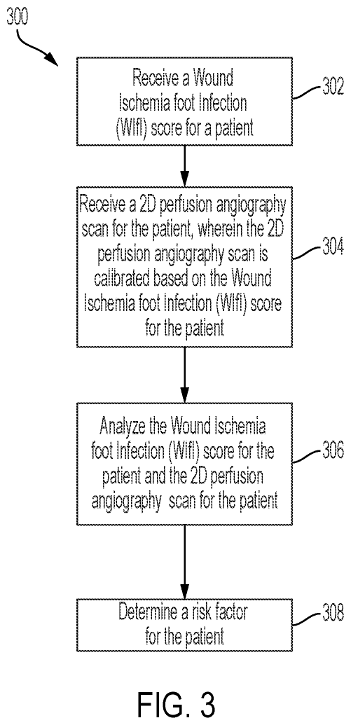

FIG. 3 illustrates a method 300 for providing healthcare intelligence and analytics according to some embodiments of the disclosure. At block 302, an information handling system may receive a Wound Ischemia foot Infection (WIfI) score for a patient. For example, the WIfI score may include Wound Ischemia foot Infection (WIfI) input(s) 202, 222, and 242. At block 304, the information handling system may receive 2-D angiography scan data for the patient, wherein the 2-D perfusion scan data is calibrated based on the Wound Ischemia foot Infection (WIfI) score for the patient. For example, the information handling system may use the WIfI score to stratify the 2-D perfusion angiography to increase precision. A stratified 2-D perfusion angiography scan may provide physicians and patients with an improved healthcare risk factor and data. In some embodiments, the 2-D perfusion angiography scan data may include 2-D perfusion angiography input(s) 204, 224, and 244, which may include a peak intensity to wound, a rate to measure baseline, a plateau to peak intensity, and/or a speed dissipation of a signal. The 2-D perfusion angiography scan may include 2-D perfusion angiography input(s) corresponding to data from the patient before and/or after percutaneous coronary intervention.

The received data at blocks 302 and 304 may be used to determine a patient outcome, which may be a prediction of a best procedure for the patient or a prediction of an outcome for a procedure on the patient. The determination may include analyzing the data at block 306 and obtaining a particular output at block 308. At block 306, the information handling system may analyze the Wound Ischemia foot Infection (WIfI) score for the patient and the 2-D perfusion angiography scan data for the patient. At block 308, the information handling system may determine a risk factor for the patient. The risk factor for the patient may include a risk factor output(s) 208, 230, and 250, which may include an onset of Critical Limb Threating Ischemia (CLTI), a baseline risk of major amputation, baseline risk of wound healing, and/or death. In some embodiments, the analysis and determination of blocks 306 and 308 may also be based on healthcare records for the patient that provide other medical data regarding the patient.

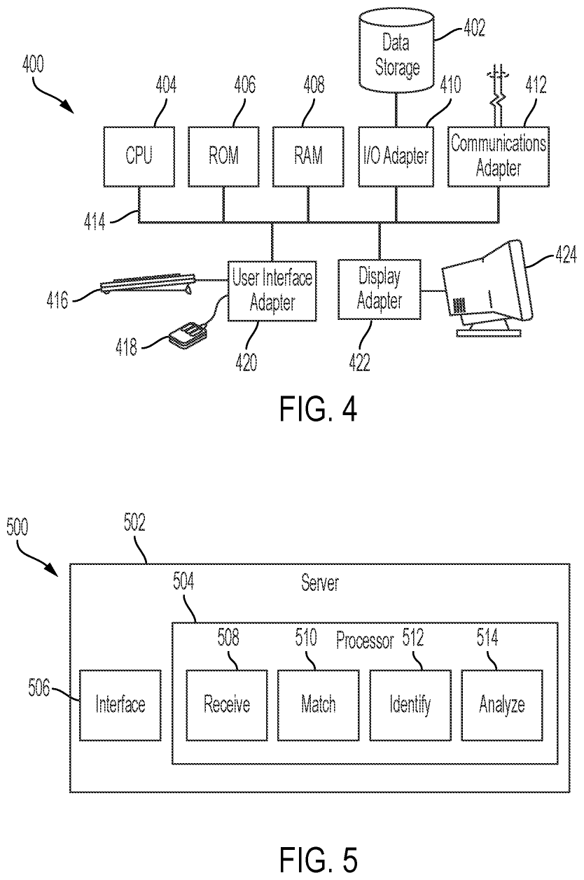

FIG. 4 illustrates an information handling system 400 such a computer system according to some embodiments of the disclosure. System 400 may include a server 102 and/or the user interface device 420. The central processing unit (CPU) 404 may be coupled to the system bus 414. The CPU 404 may be a general-purpose CPU, microprocessor, or the like. In some embodiments, a processing unit may not be limited to a CPU, and the processing unit may support the algorithm, modules, applications, and operations as disclosed. The CPU 404 may execute the algorithm or logical instructions according to some of the embodiments disclosed. The information handling system 400 may include Random Access Memory (RAM) 408, which may be SRAM, DRAM, SDRAM, or the like. The information handling system 400 may use RAM 408 to store the various data structures used by a software application configured for providing healthcare intelligence and analytics. The information handling system 400 may include Read Only Memory (ROM) 406 which may be PROM, EPROM, EEPROM, optical storage, or the like. The ROM may store information for the information handling system 400, and the RAM 408 and ROM 406 may hold user and information handling system 400 data such as healthcare data and management data.

The information handling system 400 may an include input/output (I/O) adapter 410, a communications adapter 412, a user interface adapter 420, and a display adapter 422. In certain embodiments, the I/O adapter 410 and/or the user interface adapter 420 may enable a user, such as a physician, to interact with the information handling system 400. In another embodiment, the display adapter 422 may display a graphical user interface related to the programmable rule(s), web services, or web-based application for providing healthcare intelligence and analytics. The I/O adapter 410 may connect to one or more data storage devices 4022, such as one or more of a hard drive, a Compact Disk (CD) drive, a floppy disk drive, a tape drive, to the information handling system 400. The communications adapter 412 may be adapted to couple the information handling system to a network, which may be one or more of a wireless link, a LAN and/or WAN, and/or the Internet. The user interface adapter 420 couples user input devices, such as a keyboard 416, a mouse 418, or the like, to the information handling system 400. The display adapter 422 may be driven by the CPU 404 to control the display on the display device 424.

FIG. 5 illustrates an information handling system 500 according to some embodiments of the disclosure. The information handling system 500 may include server 502, which may be configured to load and operate programmable rule(s) for receive 508, match 510, identify 512, and/or analyze 514 operations. In some embodiments, the programmable rule(s) may be operated external to the processor 504 or in another comparable device such as an embedded controller. In another embodiment, the information handling system 500 may include hardware modules configured with analog or digital logic, firmware executing FPGAs, or the like configured for receiving a plurality of healthcare data 508, matching 510 healthcare records for a same or similar patient from multiple sources, identifying 512 trends in the healthcare records (such as by inputting the matching 510 healthcare records to a machine learning algorithm), and analyzing 514 the healthcare record to obtain a particular determination. After analysis, the information handling system may determine a risk factor for the patient, which may include risk factor output(s) 208, 230, and 250, which may include an onset of Critical Limb Threating Ischemia (CLTI), a baseline risk of major amputation, baseline risk of wound healing, and/or death.

In certain embodiments, the information handling system 500 may display on a user interface the risk factor, the Wound Ischemia foot Infection (WIfI) score, the 2-D perfusion angiography scan data, and/or the healthcare records for the patient. The information handling system may include an interface 506, such as an I/O adapter 410, a communications adapter 412, a user interface adapter 420, or the like.

Although the present disclosure and certain representative advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. For example, although processors are described throughout the detailed description, aspects of the invention may be applied to the design of or implemented on different kinds of processors, such as graphics processing units (GPUs), central processing units (CPUs), and digital signal processors (DSPs). As another example, although processing of certain kinds of data may be described in example embodiments, other kinds or types of data may be processed through the methods and devices described above.

Any suitable processor-based device may be utilized including, without limitation, personal data assistants (PDAs), computer game consoles, and multi-processor servers. Moreover, the present embodiments may be implemented on application specific integrated circuits (ASIC) or very large scale integrated (VLSI) circuits. In fact, persons of ordinary skill in the art may utilize any number of suitable structures capable of executing logical operations according to the disclosed embodiments. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

Various features and advantageous details are explained more fully with reference to the non-limiting embodiments that are illustrated in the accompanying drawings and detailed in the following description. Descriptions of well-known starting materials, processing techniques, components, and equipment are omitted so as not to unnecessarily obscure the invention in detail. It should be understood, however, that the detailed description and the specific examples, while indicating embodiments of the invention, are given by way of illustration only, and not by way of limitation. Various substitutions, modifications, additions, and/or rearrangements within the spirit and/or scope of the underlying inventive concept will become apparent to those having ordinary skill in the art from this disclosure.

In the following description, numerous specific details are provided, such as examples of programming, software modules, software applications, user selections, network transactions, database queries, database structures, hardware modules, hardware circuits, hardware chips, etc., to provide a thorough understanding of disclosed embodiments. One of ordinary skill in the art will recognize, however, that embodiments of the invention may be practiced without one or more of the specific details, or with other methods, components, materials, and so forth. In other instances, well-known structures, materials, or operations are not shown or described in detail to avoid obscuring aspects of the invention.

What is claimed is:

1. A method, comprising:
receiving, by an information handling system, a Wound Ischemia foot Infection (WIfI) score for a patient;
receiving, by the information handling system, 2-D perfusion angiography scan data for the patient; and
determining, by the information handling system, a risk factor for the patient based on the 2-D perfusion angiography scan data and the Wound Ischemia foot Infection (WIfI) score using a machine learning algorithm.

2. The method of claim 1, further comprising:
receiving, by the information handling system, a healthcare record for the patient,
wherein the step of determining, by the information handling system, the risk factor for the patient is also based on the healthcare record.

3. The method of claim 2, wherein receiving, by the information handling system, the healthcare record for the patient comprises receiving at least one of clinical conditions or classifications of the patient.

4. The method of claim 1, wherein determining, by the information handling system, the risk factor comprises determining at least one of a peak intensity to wound, a rate to measure baseline, a plateau at peak intensity, or a speed dissipation of a signal.

5. The method of claim 4, wherein the 2-D perfusion angiography scan data for the patient corresponds to the patient prior to a percutaneous coronary intervention.

6. The method of claim 4, wherein the 2-D perfusion angiography scan data for the patient corresponds to the patient after a percutaneous coronary intervention.

7. The method of claim 1, wherein determining, by the information handling system, the risk factor for the patient comprises determining a risk of least one of an onset of Critical Limb Threating Ischemia (CLTI), a major amputation, a wound healing, or a death.

8. An information handling system, comprising:
a memory; and
a processor coupled to the memory, in which the processor is configured to perform steps comprising:
receiving a Wound Ischemia foot Infection (WIfI) score for a patient;
receiving 2-D perfusion angiography scan data for the patient; and
determining, using a machine learning algorithm, a risk factor for the patient based on the 2-D perfusion angiography scan data and the Wound Ischemia foot Infection (WIfI) score.

9. The information handling system of claim 8, wherein the processor is further configured to perform steps comprising:
receiving a healthcare record for the patient,
wherein the step of determining, by the information handling system, the risk factor for the patient is also based on the healthcare record.

10. The information handling system of claim 9, wherein receiving, by the information handling system, the healthcare record for the patient comprises receiving at least one of clinical conditions or classifications of the patient.

11. The information handling system of claim 8, wherein the step of determining the risk factor for the patient comprises determining at least one of a peak intensity to wound, a rate to measure baseline, a plateau at peak intensity, or a speed dissipation of a signal.

12. The information handling system of claim 11, wherein the 2-D perfusion angiography scan data for the patient corresponds to the patient prior to a percutaneous coronary intervention.

13. The information handling system of claim 11, wherein the 2-D perfusion angiography scan data for the patient corresponds to the patient after a percutaneous coronary intervention.

14. The information handling system of claim 8, wherein determining, by the information handling system, the risk factor for the patient comprises determining a risk of least one of an onset of Critical Limb Threating Ischemia (CLTI), a major amputation, a wound healing, or a death.

15. A computer program product comprising:
a non-transitory computer readable medium comprising instructions for causing an information handling system to perform steps comprising:
receiving a Wound Ischemia foot Infection (WIfI) score for a patient;
receiving 2-D perfusion angiography scan data for the patient; and
determining, by the information handling system, a risk factor for the patient based on the 2-D perfusion angiography scan data and the Wound Ischemia foot Infection (WIfI) score using a machine learning algorithm.

16. The computer program product of claim 15, wherein the non-transitory computer readable medium further comprises instructions for:
receiving a healthcare record for the patient,
wherein the step of determining, by the information handling system, the risk factor for the patient is also based on the healthcare record.

17. The computer program product of claim 16, wherein receiving, by the information handling system, the healthcare record for the patient comprises receiving at least one of clinical conditions or classifications of the patient.

18. The computer program product of claim 15, wherein determining, by the information handling system, the risk factor comprises determining at least one of a peak intensity to wound, a rate to measure baseline, a plateau at peak intensity, or a speed dissipation of a signal.

19. The computer program product of claim 18, wherein the 2-D perfusion angiography scan data for the patient corresponds to the patient prior to a percutaneous coronary intervention.

20. The computer program product of claim 15, wherein determining, by the information handling system, the risk factor for the patient comprises determining a risk of least one of an onset of Critical Limb Threating Ischemia (CLTI), a major amputation, a wound healing, or a death.
